(19) United States
(12) Patent Application Publication
(10) Pub. No.: US 2004/0214148 A1
(43) Pub. Date: Oct. 28, 2004

(54) Updating health care protocols

(76) Inventors: Robert J. Salvino, Seattle, WA (US); Richard C. Nova, Kirkland, WA (US)

Correspondence Address: SHUMAKER & SIEFFERT, P. A., 8425 SEASONS PARKWAY, SUITE 105, ST. PAUL, MN, 55125, US

(21) Appl. No.: 10/818,016

(22) Filed: Apr. 5, 2004

Related U.S. Application Data
(60) Provisional application No. 60/464,859, filed on Apr. 22, 2003.

(51) Int. Cl. G09B 23/28; A61N 1/24

(52) U.S. Cl. 434/262; 600/300

(57) ABSTRACT

The invention is directed to techniques for managing health care protocols with a medical device such as a defibrillator, patient monitor, trainer, or other device. In particular, the invention is directed to techniques for updating the health care protocols, which may involve the use of recorded storage media. The recorded storage medium may be connected to the medical device via an adapter, such as a disk drive. Alternatively, the data storage medium and a medical device battery may be included within the same device. In other implementations of the invention, the medical device receives an updated health care protocol from another medical device. The communication link between the medical devices may be configured in a number of ways. For example, medical devices may communicate health care protocols via a physical communication link, a wireless communication link, or a radio frequency communication link.

### TECHNICAL FIELD

The invention relates to patient health care protocols, and more particularly, to protocols implemented with the assistance of electronic devices.

### BACKGROUND

Emergency medical technicians (EMTs) save lives every day by responding to emergencies. EMTs provide immediate medical attention to a patient. Medical attention may include, for example, determining the nature and extent of the condition of the patient and administering therapy.

Jurisdictions generally recognize degrees of proficiency among EMTs. Some EMTs are trained and qualified to provide an extensive range of pre-hospital care, and others are trained and qualified to provide lesser degrees of care. In general, EMTs having more advanced training may administer intravenous fluids, use a manual defibrillator to restore a normal heart rhythm, and apply advanced airway techniques. Qualified paramedics may provide extensive care, such as performing an endotracheal intubation, administering medications and interpreting electrocardiograms.

Various jurisdictions hold EMTs subject to strict rules and guidelines pertaining to appropriate emergency care. The rules and guidelines differ from jurisdiction to jurisdiction.

### SUMMARY

The invention is directed to techniques for managing health care protocols with a medical device such as a defibrillator, patient monitor, trainer, or other device. The device may be brought to the site of a patient in need of medical assistance, or may be used to train personnel such as EMTs, or both. In particular, the invention is directed to techniques for updating the health care protocols. The invention includes, but is not limited to, the techniques described herein.

For purposes of the invention, “protocol” is defined broadly, and encompasses plans, procedures and rules for treating patients. The term encompasses general procedures, as well as procedures applicable to a specific patient complaint, condition or presentation. “Protocol” further includes rules and guidelines applicable to a jurisdiction. “Protocol” also includes training health care protocols. The information presented pursuant to the protocol may include sets of procedures, reference information, utilities such as calculators, timers and written, graphical or audible prompts to the operator. Additional aspects of a “protocol,” as the term is used herein, will be described below.

From time to time, a regulating authority may change one or more health care protocols, and it is important to change the protocols stored in medical devices to reflect the change. One technique for updating protocols in medical devices involves receiving the protocol on recorded storage media, such as data card, compact disk or floppy disk. In one implementation, the storage medium may be included with a battery, so replacement of the device's power supply results in an update of stored protocols.

Another technique involves having a medical device receive an updated health care protocol from another medical device. The communication link between the medical devices may be established in a number of ways. The medical devices may communicate via a physical communication link, for example, or a wireless communication link.

In one embodiment, the invention presents a method by which one medical device may receive an update from another medical device. The method comprises establishing a communication link with a medical device, receiving a first health care protocol from the medical device, and updating a second health care protocol based on the first health care protocol.

In another embodiment, the invention is directed to a method in which a medical device receives an update from a recorded storage medium. The method comprises receiving the recorded storage medium, receiving a first health care protocol from the recorded storage medium, and updating a second health care protocol based on the first health care protocol.

The invention further includes computer-readable media comprising instructions for causing a programmable processor to carry out the methods described above.

In a further embodiment, the invention is directed to an apparatus comprising a battery and a recorded storage medium that stores at least a part of a health care protocol. The apparatus may be configured to mate to a defibrillator, a patient monitor, or a trainer, for example.

In an added embodiment, the invention presents a medical device comprising an input device to receive recorded storage medium and to retrieve a health care protocol stored on the medium. The medical device also includes a memory to store the health care protocol.

The invention may have one or more advantages. Emergency medical personnel may respond to a wide variety of emergencies, and it may be difficult to remember the protocols for all of the situations the personnel may encounter. The invention helps guide the emergency medical personnel through the established procedures of an applicable protocol. Moreover, the invention providing techniques for updating protocols for medical devices, including trainers, medical devices can help emergency medical personnel apply the proper and/or current protocols.

Updating a health care protocol by various means allows a medical device to be flexible and conform to protocols of different jurisdictions. As the protocols of different jurisdictions change, the protocols stored in a plurality of medical devices can be readily updated. Moreover, the updates to a health care protocol may be supplied to a medical device via many paths.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a schematic view of a defibrillator that may be used to practice the techniques of the invention.

FIG. 2 is a schematic view of a patient monitor that may be used to practice the techniques of the invention.

FIG. 3 is a flow diagram illustrating a technique for selection of a protocol and presentation of information pursuant to the selected protocol, according to an embodiment of the invention.

FIG. 4 is an example of a screen display that lists protocols supported by a device such as a patient monitor or defibrillator.

FIG. 5 is an example of a screen display that presents information pursuant to a protocol, including procedures and a prompt for an operator acknowledgement.

FIG. 6 is an example of a screen display that presents information pursuant to a protocol, including reference information and a field for information entry.

FIG. 7 is an example of a screen display that presents information pursuant to a protocol, including a calculator utility and prompts for an operator to report the status of therapy.

FIGS. 8 through 10 are examples of screen displays that present information pursuant to a protocol, and illustrate branching according to an embodiment of the invention.

FIG. 11 is an example of a screen display that presents information pursuant to a protocol, including prompts for setting a timer.

FIG. 12 is an example of a screen display of an event log.

FIG. 13 is a schematic diagram of a system for customizing a protocol according to an embodiment of the invention, including a server and one or more client devices.

FIG. 14 is a flow diagram illustrating techniques employed by the server and client devices for preparing and storing customized protocols according to an embodiment of the invention.

FIGS. 15A-15C are schematic diagrams illustrating exemplary techniques for updating a health care protocol of a medical device.

FIG. 16 is a schematic diagram illustrating additional exemplary techniques for updating a health care protocol of one or more medical devices.

FIG. 17 is a flow diagram illustrating an exemplary technique for using a recorded storage medium to update a health care protocol.

FIG. 18 is a flow diagram illustrating an exemplary technique for communicating an updated health care protocol between or among medical devices.

### DETAILED DESCRIPTION

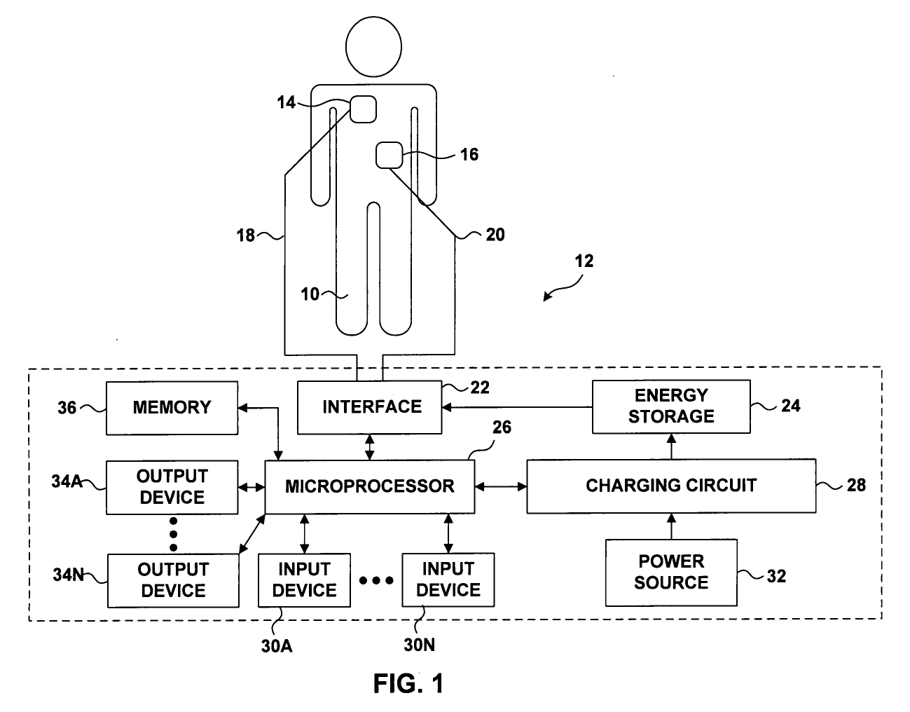

FIG. 1 is a block diagram showing a patient 10 coupled to an external defibrillator 12. External defibrillator 12 is one example of a device that may be used to practice the invention. Defibrillator 12 administers defibrillation therapy to patient 10 via electrodes 14 and 16, which may be hand-held electrode paddles or adhesive electrode pads placed on the skin of patient 10. The body of patient 10 provides an electrical path between electrodes 14 and 16.

Electrodes 14 and 16 are coupled to defibrillator 12 via conductors 18 and 20 and interface 22. In a typical application, interface 22 includes a receptacle, and connectors 18, 20 plug into the receptacle. Electrical impulses or signals may be sensed by defibrillator 12 via electrodes 14 and 16 and interface 22. Electrical impulses or signals may also be delivered from defibrillator 12 to patient 10 via electrodes 14 and 16 and interface 22.

Interface 22 includes a switch (not shown in FIG. 1) that, when activated, couples an energy storage device 24 to electrodes 14 and 16. Energy storage device 24 stores the energy for a dosage of energy or current to be delivered to patient 10. The switch may be of conventional design and may be formed, for example, of electrically operated relays. Alternatively, the switch may comprise an arrangement of solid-state devices such as silicon-controlled rectifiers or insulated gate bipolar transistors.

Energy storage device 24 includes components, such one or more capacitors, that store the energy to be delivered to patient 10 via electrodes 14 and 16. Before a defibrillation pulse may be delivered to patient 10, energy storage device 24 must be charged. A microprocessor 26 directs a charging circuit 28 to charge energy storage device 24 to a high voltage level. Microprocessor 26 may automatically direct charging circuit 28 to begin charging, or microprocessor 26 may direct charging circuit 28 to begin charging upon the instruction of an operator such as an EMT. An operator may instruct microprocessor with one or more input devices 30A-30N (hereinafter 30), such as one or more buttons, a keyboard, a touch screen, a voice recognition module or a pointing tool.

Charging circuit 28 comprises, for example, a flyback charger that transfers energy from a power source 32 to energy storage device 24. Because the life of patient 10 may depend upon receiving defibrillation, charging should take place rapidly so that the defibrillation shock may be delivered with little delay. Power source 32 may comprise a replaceable battery. As will be discussed below, the invention supports techniques whereby installation of a new battery causes defibrillator 12 to receive a recorded storage medium at the same time.

When the energy stored in energy storage device 24 reaches the desired level, defibrillator 12 is ready to deliver the defibrillation shock. The shock may be delivered automatically or manually. Defibrillator 12 may notify the operator that charging is competed using one or more output devices 34A-34N (hereinafter 34), such as a display screen, an audible sound generator, a voice synthesizer, a printer or an indicator light. In the case of a manual delivery, microprocessor 26 may activate an output device 34 that informs the operator that defibrillator 12 is ready to deliver a defibrillation shock to patient 10. The operator may activate the switch by manually operating an input device 30, such as by pressing a button. Defibrillator 12 delivers a defibrillation shock to patient 10.

The goal of defibrillation is to depolarize the heart with electrical current and cause the heart to reestablish a normal sinus rhythm. In some patients, one shock is insufficient to reestablish normal rhythm, and one or more additional defibrillation shocks may be required. Before another shock may be administered, however, charging circuit 28 ordinarily must transfer energy from power source 30 to energy storage device 24, thereby recharging energy storage device 24. In recharging energy storage device 24, as in the initial charging, time is of the essence, and charging circuit 28 therefore charges energy storage device 24 quickly. The energy or current dosage delivered to patient 10 need not be the same in each shock.

Power source 30 may comprise, for example, batteries and/or an adapter to an exterior power source such as an electrical outlet. In addition to supplying energy to charging circuit 28 and energy storage device 24, power source 30 also supplies power to components such as microprocessor 26, input devices 30 and output devices 34, e.g., via a power supply circuit (not shown in FIG. 1).

In addition to controlling the delivery of a defibrillation pulse, microprocessor 26 may also modulate the electrical pulse delivered to patient 10. Microprocessor 26 may, for example, regulate the shape of the waveform of the electrical pulse and the duration of the pulse. Microprocessor 26 may also monitor electrocardiogram (ECG) signals sensed via electrodes 14 and 16 and received via interface 22. Microprocessor 26 may display these signals via an output device 34 such as a display screen or printer. In some embodiments, microprocessor 26 may also analyze the ECG signals and determine whether patient 10 suffers from a condition that requires a defibrillation shock. In addition, microprocessor 26 may evaluate the efficacy of an administered defibrillation shock.

Furthermore, microprocessor 26 may store and retrieve data from memory 36. Memory 36 may include volatile storage, such as random access memory, and/or non-volatile storage, such as Flash memory or a hard disk. Memory 36 stores instructions that direct the operation of microprocessor 26. In addition, memory 36 stores information about patient 10 and defibrillator 12. For example, memory 36 may store the ECG of patient 10, information about the number of shocks delivered to patient 10, the energy delivered per shock, the timing of shocks and the patient response to shocks.

In addition, memory 36 stores health care protocols that pertain to various medical situations. As used herein, “protocol” is defined broadly. “Protocol” encompasses procedures for treating patients, including plans, guidelines and rules for treating patients. The term encompasses general procedures, as well as procedures applicable to a specific patient complaint, condition or presentation. “Protocol” further includes rules and guidelines applicable to a jurisdiction, such as treatment procedures adopted by a regulating authority responsible for overseeing EMTs. A regulating authority may be, for example, a regional health care system administrator or a government administrative agency that licenses and regulates EMTs.

The procedures of a protocol may be embodied as a checklist, a questionnaire, a flow diagram, a series of notices or the like. As used herein, “protocol” includes the embodiment of the procedures, as well as what an operator should do in carrying out the procedures. In one protocol, for example, an operator may be given considerable discretion in attending to patient 10. In another protocol, by contrast, the operator may be required to perform an established sequence of actions, with little left to the discretion of the operator. “Protocol” further includes procedures geared to the training and certification of the operator. A protocol for cardiopulmonary resuscitation (CPR) administered by a paramedic, for example, may differ from a protocol for CPR administered by a police officer.

Some protocols may include diagnosis. For example, a protocol may involve collecting information about the exhibited symptoms, complaints, medical history and vital signs of the patient. On the basis of the collected information, the protocol may direct that the patient will be suspected of suffering from a particular condition, and will be treated accordingly. Another protocol may pertain to the diagnosed condition or the treatment for the diagnosed condition.

As used herein, “protocol” also includes sub-protocols that may be used in comprehensive protocols. A sub-protocol pertaining to the application of leads to measure an ECG, in one example, is included in a protocol applicable to a patient complaining of heart pain, or a protocol applicable to an unconscious patient. The sub-protocol may be exactly the same in both protocols, even though the protocols are directed to different situations.

The term “protocol” also encompasses recording and record keeping that accompanies attending to patient 10. When a protocol calls for a specific sequence of actions, for example, the protocol includes recording each action taken and documenting how each action was performed. By contrast, when a protocol calls for a routine or non-specific action, the protocol might not require recording the details of how the action was performed. Memory 36 may maintain an “event log,” which records actions in the course of attending to patient 10.

In some protocols, timing of treatment is important. For example, a protocol may not only specify what drugs are to be administered and in what amounts, but also the timing of the dosages. Accordingly, the term “protocol” also includes schedules and timers for administering treatment. An example of an application involving a timer will be provided below.

“Protocol” further includes display modes. The term “display modes” refers to sets of data that are significant to the patient's condition, and that may be important in administering the protocol. In a stroke display mode, for example, data pertaining to oxygen saturation of blood flowing to the brain, temperature of the body, and electrical activity in the brain may be displayed. In a cardiac display mode, by contrast, information pertaining to the heart rate, heart rhythm and condition of the heart may be featured. Some data may be featured in more than one display mode. For example, heart rate may be included in several different display modes.

“Protocol” further includes references and utilities for carrying out procedures for diagnosis and treatment of a patient. The term “references” includes any information that may assist in attending to the patient, such as formulas or information about medications patient 10 may be taking. The term “utilities” includes any tools that may assist in treating patient 10. An example of a utility may be a calculator that computes dosages of medication, or converts English units to metric units and vice versa.

“Protocol” also includes the instructions used by a processor such as microprocessor 26 to present information to assist an operator attending patient 10. A protocol may be embodied as a computer-readable medium comprising instructions for a processor. A “computer-readable medium” includes but is not limited to read-only memory, Flash memory and a magnetic or optical storage medium. The medium may comprise instructions for causing a programmable processor to present information in a variety of formats, and to interact with an operator in many ways. As will be described below, a protocol may be represented as a set of linked objects, but the invention is not limited to this implementation or to any particular programming technique.

“Protocol” further includes a training health care protocol. A “training health care protocol” provides a simulation of a protocol that would be applied in an actual emergency. A training protocol can be used by a trainer, i.e., a device that mimics the performance of a working defibrillator or other medical device, but that does not have full monitoring or therapy capability. In addition, a training protocol can be used by a working defibrillator or other medical device in a “training mode,” i.e., a mode in which the defibrillator or medical device simulates performance in an actual emergency. By training with a medical device implementing one or more training protocols, an EMT can become familiar with protocols applied in real emergencies.

In defibrillator 12, memory 36 stores one or more protocols pertaining to defibrillation therapy. A defibrillation therapy protocol may, for example, require an operator to perform certain tasks before defibrillation is attempted, such as opening the airway of patient 10 and checking for proper breathing. The protocol may also include restrictions concerning the number of defibrillation shocks that may be administered, the timing of the shocks, the amount of energy delivered in the shocks, and so forth.

Different jurisdictions may require different defibrillation therapy protocols. In a typical application, memory 36 stores the protocol for the jurisdiction in which defibrillator 12 is used. Memory 36 may also store other defibrillation therapy protocols as well. Memory 36 may further store protocols in addition to defibrillation therapy protocols. These additional protocols may pertain to identification of and therapy for conditions such as closed head injury, stroke, or various forms of trauma such as bleeding, broken bones or burns. In other words, the protocols stored in memory 36 of defibrillator 12 need not be limited to defibrillation therapy.

Microprocessor 26 of defibrillator 12 selects a protocol as a function of patient data. In particular, microprocessor 26 receives data concerning patient 10 from an operator via input device 30 or from data collected via electrodes 14, 16. For example, microprocessor 26 may receive data concerning the age, sex and weight of patient 10, entered by the operator via an input device 30, such as a keyboard. The operator may also enter information pertinent to the medical history of patient 10, such as whether the patient has a history of heart problems, hypertension, epilepsy, and so forth. Microprocessor 26 may also receive information concerning the heart rate and heart rhythm of patient 10 via electrodes 14, 16. In addition, microprocessor 26 may receive data concerning patient 10 from other sources, such as another medical device. Patient data from other sources may include, for example, blood pressure data, blood glucose levels, an electroencephalogram, and the like.

Microprocessor 26 selects a protocol as a function of the received patient data. When patient 10 exhibits ventricular fibrillation, for example, microprocessor 26 may select a defibrillation therapy protocol. When patient 10 has collapsed into unconsciousness but does not exhibit problems with heart rhythm as sensed via electrodes 14, 16, microprocessor 26 may select another protocol, such as a stroke protocol.

The selected protocol will assist an operator, such as an EMT, attending to patient 10. Upon selection of a protocol, microprocessor 26 presents information pursuant to the selected protocol. The information may be presented using one or more output devices 34. Examples of information that may be presented in a defibrillation therapy protocol will be provided below.

By presenting information pursuant to a protocol, defibrillator 12 guides the operator through the procedures of the protocol. Defibrillator 12 may track and record whether and how the procedures were followed. Defibrillator 12 may, for example, present the operator with one or more tasks to be performed pursuant to the protocol and may prompt the operator to perform the tasks. Defibrillator 12 may thereafter receive an acknowledgement from the operator that the tasks have been performed. The operator may be required, for example, to activate an input device 30 to acknowledge that prescribed procedures have been carried out. The performance of the procedures, and other data such as the time of acknowledgment entered by the operator, may be recorded in memory 36.

Recordation may serve many functions. Recordation may assist a hospital in the compilation of a medical history for patient 10, for example, or may provide evidence showing that established procedures were followed. An operator such as an EMT may use the recorded data in preparing a “run report” that documents the emergency. The recorded data may also be helpful to the regulating authority that establishes protocols when analyzing whether established protocols could be made more effective.

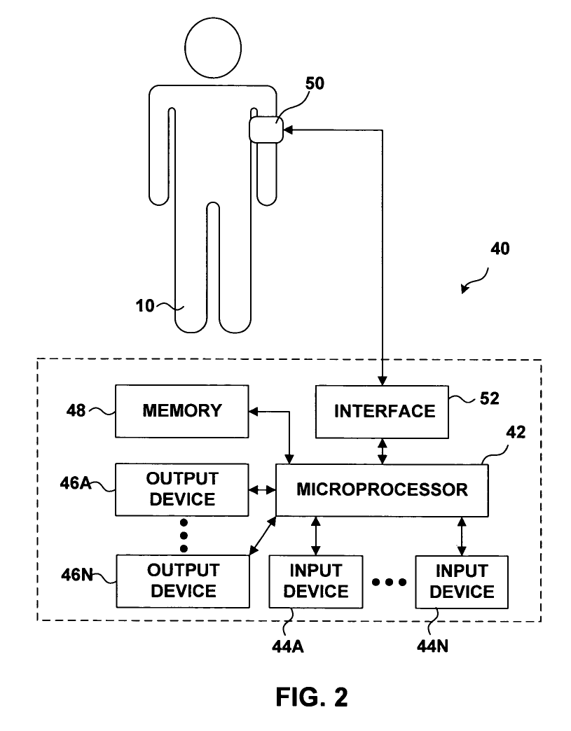

Defibrillator 12 is an example of a medical device that can practice the invention. Another example of such a device is a patient monitor 40, which is shown in FIG. 2. Patient monitor 40 is similar to defibrillator 12 in that patient monitor 40 includes a microprocessor 42, input devices 44A-44N (hereinafter 44), output devices 46A-46N (hereinafter 46) and memory 48. A further example of a medical device is a trainer that simulates the performance of a defibrillator or other medical device. The trainer may mimic the appearance and operation of the medical device, without actually providing therapy to a patient.

Unlike defibrillator 12, however, patient monitor 40 does not include apparatus for delivering therapy to patient 10. Rather, patient monitor 40 includes a monitoring device 50, which is coupled to patient monitor 40 via interface 52. Monitoring device 50 may be any device that detects, monitors or measures any characteristic of patient 10. Monitoring device 50 may be, but need not be, proximate to patient 10 or in contact with patient 10.

In FIG. 2, monitoring device 50 is depicted as a blood pressure cuff, but monitoring device 50 may be any of several monitoring devices, such as a temperature sensor, a blood oxygen meter, a carbon dioxide sensor, and the like. A multi-lead set of electrodes, such as a twelve-lead apparatus for sensing electrical signals, is also an example of a monitoring device. An electrode pair, like electrodes 14 and 16 of defibrillator 12, is another example of a monitoring device.

Memory 48 stores protocols that pertain to different conditions or complaints of patient 10. Microprocessor 42 selects a protocol as a function of patient data. Microprocessor 42 may receive patient data from any of several sources. Patient data may be entered by an operator using input device 44, for example, data collected via monitoring device 50, or received from another medical device. Microprocessor 42 selects a protocol as a function of the received patient data.

The selected protocol will assist an operator attending to patient 10. Microprocessor 42 presents information pursuant to the selected protocol using one or more output devices 46 and guides the operator through the procedures of the protocol. Patient monitor 40 may track and record whether and how the procedures were followed.

Although patient monitor 40 does not include an apparatus for performing defibrillations, patient monitor 40 may include a defibrillation therapy protocol. In some cases, an operator responding to an emergency may not carry a defibrillator capable of selecting a protocol and presenting information pursuant to the selected protocol, like defibrillator 12 in FIG. 1. In these cases, the operator may be guided by the defibrillation therapy protocol stored in memory 48 of patient monitor 40.

Patient monitor 40 may include a power source (not shown), such as a replaceable battery. In an embodiment of the invention discussed below, the invention supports techniques whereby installation of a new battery causes a medical device such as patient monitor 40 to receive a recorded storage medium.

Defibrillator 12 and patient monitor 40 are examples of devices that may be used to practice the invention, but the invention is not limited to practice with such devices. The techniques of the invention may be adapted to any of several medical devices that diagnose, monitor or provide therapy to patient 10. The techniques of the invention may also apply to a stand-alone device that provides no diagnosis, monitoring or therapy.

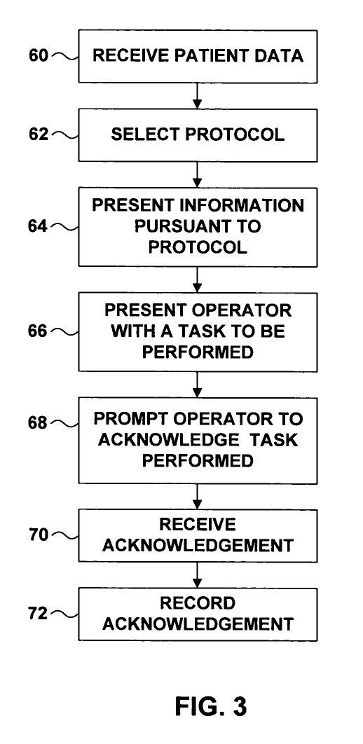

FIG. 3 is a flow diagram illustrating an example of a technique for selection of a protocol and presentation of information pursuant to the selected protocol. A processor in a device, such as microprocessor 26 in defibrillator 12 or microprocessor 42 in patient monitor 40, receives patient data (60). As noted above, patient data may be received from any of several sources, such as data entered by an operator or data received by a monitoring device. The data may pertain to the current physical or mental condition of the patient, the complaints of the patient, a medical history, measured aspects of medical significance such as heart rate, body temperature or blood pressure, and the like.

The processor selects a protocol as a function of the received patient data (62). The protocol may be a defibrillation therapy protocol when the patient data indicates the patient is in need of defibrillation, for example, or a stroke protocol when available data suggests the patient has suffered a stroke. The processor presents information via one or more output devices pursuant to the selected protocol (64). Examples of presented information will be discussed below. The presented information guides the operator attending to the patient.

In one embodiment of the invention, the device may monitor and record actions taken pursuant to the protocol. FIG. 3 illustrates an example of monitoring and recording. The device presents an operator with a task to be performed pursuant to a protocol (66) and prompts the operator to acknowledge that the task has been performed (68). The device receives the acknowledgement (70) and may record the acknowledgement (72).

For example, a medical device may notify operator to administer a dosage of medication as part of a protocol. The medical device may further prompt the operator to enter an acknowledgement that administration of the dosage has been accomplished. The device may record the administration of the dosage as an “event” in an event log. The device may also record other matters pertaining to the dosage, such as the drug administered, the concentration, the mode of administration (such as drip or bolus) and the time of administration. If the drug is one that should be administered at specified intervals according to a protocol, the device may also activate a timer that will prompt the operator to administer the drug at those intervals.

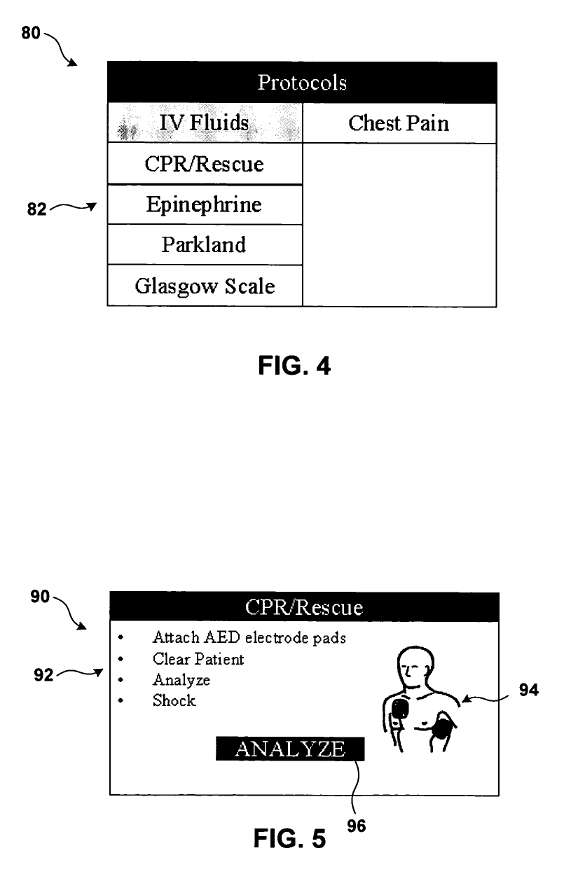

FIG. 4 is an exemplary screen display 80 listing various possible protocols or classifications of protocols supported by a device such as a patient monitor or defibrillator. Protocols displayed in FIG. 4 include one or more protocols for administration of intravenous (IV) fluids, include one or more protocols for CPR/rescue, include one or more protocols for bolus drug administration such as administration of epinephrine, include one or more protocols for trauma such as the protocols set out in the Parkland Trauma Handbook, one or more coma protocols such as a protocol using the Glasgow Coma Scale to assess coma severity, and one or more protocols directed to a patient complaint such as chest pain.

A medical device may support more or fewer protocols than are shown in FIG. 4, or different protocols than are shown in FIG. 4. Other possible protocols may include protocols for administering certain medications such as adenosine, atropine or dopamine. Protocols may also pertain to procedures for therapy or monitoring, such as intubating the patient or performing an electroencephalogram, or to patient complaints such as vertigo or numbness. A device may also support a generic protocol, which may be applicable when the more specialized protocols are inapplicable.

In one embodiment of the invention, an operator may enter patient data that directs a medical device to select a particular protocol or classification of protocols. Screen display 80 represents a menu 82 of device-supported protocols. In the case of a patient complaining of chest pain, for example, the operator may select “chest pain” from menu 82, thereby instructing the device to select a protocol consistent with the complaint of the patient. In other words, the device receives data concerning a patient in the form of an express operator identification of the physical or mental condition of the patient, and the device selects a protocol applicable to the condition.

Selection of a protocol as a function of a specific direction by an operator may be called an “operator invoked” or “user invoked” protocol selection. An operator invoked protocol selection may be performed at any time. An operator may, for example, direct the device to change from one protocol to another protocol in the course of attending to the patient.

In some embodiments of the invention, an operator invoked protocol selection may cause the device to display a sub-menu prompting the operator for a more specific protocol identification. For example, when an operator directs the device to select a trauma protocol, the device may present the operator with a sub-menu that prompts the operator to specify the general nature of the trauma, so that the device may select the applicable protocol.

A device may also support “event initiated” protocol selection, in which the device selects a protocol based upon an event. Patient data collected from one or more monitoring devices or operator input may generate an event that results in a protocol selection. An event initiated protocol selection may also be performed at any time. When a patient is being treated according to a stroke protocol and the device detects that the patient has gone into cardiac arrest, for example, the device may automatically switch from stroke protocol to CPR/rescue protocol.

Selection of a sub-protocol is another example of an event initiated protocol selection. A device may be presenting information pursuant to a protocol such as a chest pain protocol, and the operator is directed to attach a set of twelve leads to the patient for a twelve-lead ECG. At this point, a twelve-lead protocol may be initiated. The twelve-lead protocol may prompt the operator to enter patient information, such as information pertaining to the age and sex of the patient, information pertaining to any history of diabetes or hypertension, and the present pain assessment of the patient. The protocol may also direct the operator to attach the leads, and may present information pertaining to actions the operator is to perform. For example, the protocol may remind the operator “Make sure patient is laying on back if possible” and “Do proper skin preparation.” Once the twelve-lead analysis is completed, the device may return to the chest pain protocol.

The same twelve-lead protocol may be a sub-protocol in protocols other than a chest pain protocol, and may be initiated by patient data pertaining to a variety of conditions or complaints. The twelve-lead protocol may also be a protocol that stands on its own and is not initiated as a sub-protocol of another protocol.

Furthermore, a device may support a protocol selection that is a hybrid of operator invoked protocol selection and event initiated protocol selection. For example, an operator, attending to a patient that may be suffering from a coma, may initiate a Glasgow Coma Scale protocol to assess the coma severity. The recorded responses of the patient to tests administered pursuant to the Glasgow Coma Scale tests may be events that initiate a particular coma protocol, based upon the severity of the coma.

FIG. 5 is an exemplary screen display 90 that may accompany a protocol such as a CPR/rescue protocol. The CPR/rescue protocol may be “operator invoked” or “event initiated.” In screen display 90, an operator is assumed to have an automated external defibrillator (AED) at hand, and screen display 90 may be displayed by the AED or by another medical device. In other words, screen display 90 may be presented on a device that performs analysis or delivers therapy or both, or screen display 90 may be presented on a device that neither performs analysis nor delivers therapy.

Screen display 90 includes procedures 92 that inform the operator of the course of action to be taken, e.g., attach the electrodes to the patient and to clear the patient. In some embodiments of the invention, the written instructions may be accompanied by a pictorial or animated presentation 94 demonstrating the tasks to be performed.

Screen display 90 also includes a prompt 96 for the operator to select when analysis of the heart rhythms of the patient may be commenced. The operator may use an input device such as a push button or a pointing device to acknowledge that analysis is ready to be performed, or that analysis is being performed automatically by the AED. The operator may be directed to administer a shock manually, or a shock may be administered automatically, if the analysis indicates that the patient has a shockable rhythm.

Procedures 92 are exemplary and other procedures may be listed. The procedures may take the operator through the course of action in greater detail, for example, by describing in detail how the electrode pads are to be removed from a pouch, separated from a liner and placed at specific locations on the bare skin of the patient. In the case of a more highly trained operator, such as a paramedic, the procedures need not be as explanatory.

In some embodiments of the invention, a device may display information pursuant to protocols appropriate to a particular of training and experience. A portable device carried by a paramedic, for example, may be present information in a fashion that may be most useful to a paramedic. In other embodiments of the invention, a device may display information pursuant to protocols appropriate to multiple levels of training and experience. A device that may be used by operators of different training and experience, such as an AED, may interrogate an operator as to level of training and experience of the operator. The device may display information pursuant to protocols appropriate to the level of training and experience reported by the operator.

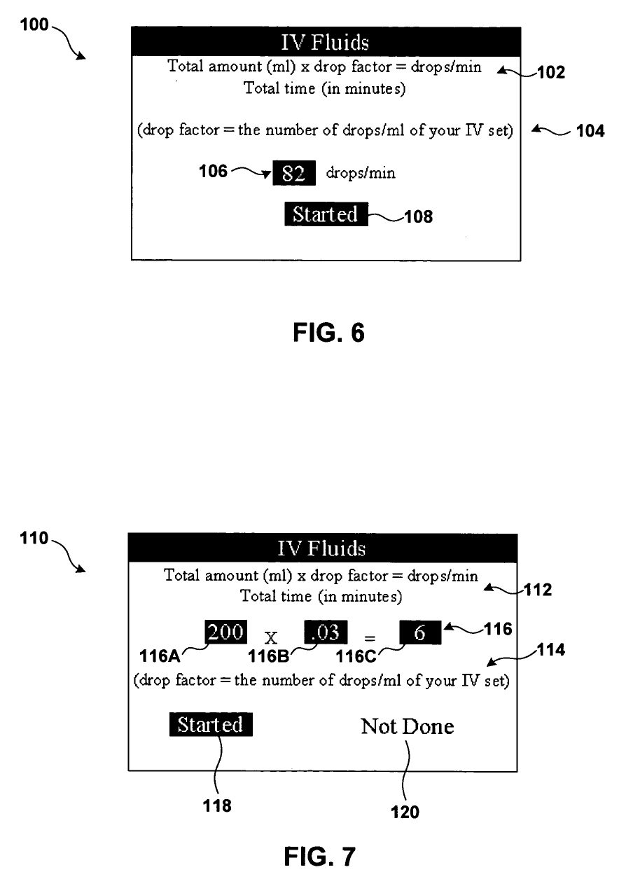

FIG. 6 is an exemplary screen display 100 that may accompany a protocol such as an IV fluids administration protocol. Screen display 100 illustrates presentation of information pursuant to the protocol. The presented information includes reference information, in the form of a formula 102 and a definition 104. Formula 102 and definition 104 may be displayed automatically to refresh the recollection of an operator, for example, or may be displayed in response to a query from the operator.

Exemplary screen display 100 also includes a field for information entry 106. Information entry field 106 may be displayed when the IV fluids protocol includes a requirement or guideline that the number of drops per minute be recorded. In some jurisdictions, the IV fluids protocol need not record the number of drops administered per minute, and in those jurisdictions, information entry field 106 may be omitted.

In the IV fluids administration protocol or other protocols, an operator may enter information using a data entry technique other than entering a number in information entry field. The operator may, for example, select an item from a menu or select a checkbox or interact with another structured data entry format.

Exemplary screen display 100 further includes a prompt 108 for the operator to select when IV fluid administration has been started. The operator may use an input device such as a push button or a pointing device to acknowledge that fluid administration has been started. Acknowledgment may cause the event log to reflect that IV fluids were administered and the time that administration began. Data entered in field for information entry 106 may be also included in the event log.

FIG. 7 is an exemplary screen display 110 that may accompany an IV fluids administration protocol. Screen display 110, like screen display 100 in FIG. 6, includes reference information in the form of a formula 112 and a definition 114. Unlike screen display 100, screen display 110 includes a utility tool 116 that may assist the operator attending to the patient. Utility tool 116 is a calculator that computes drops per minute as a function of data entered by the operator into information entry fields 116A and 116B. The operator may use an input device such as a keyboard or pointing device to enter numbers into information entry fields 116A and 116B. The result of the computation appears in field 116C.

Screen display 110, unlike screen display 100, includes two prompts 118, 120 for the operator to report the status of fluid administration. The operator may select the status that best describes the situation.

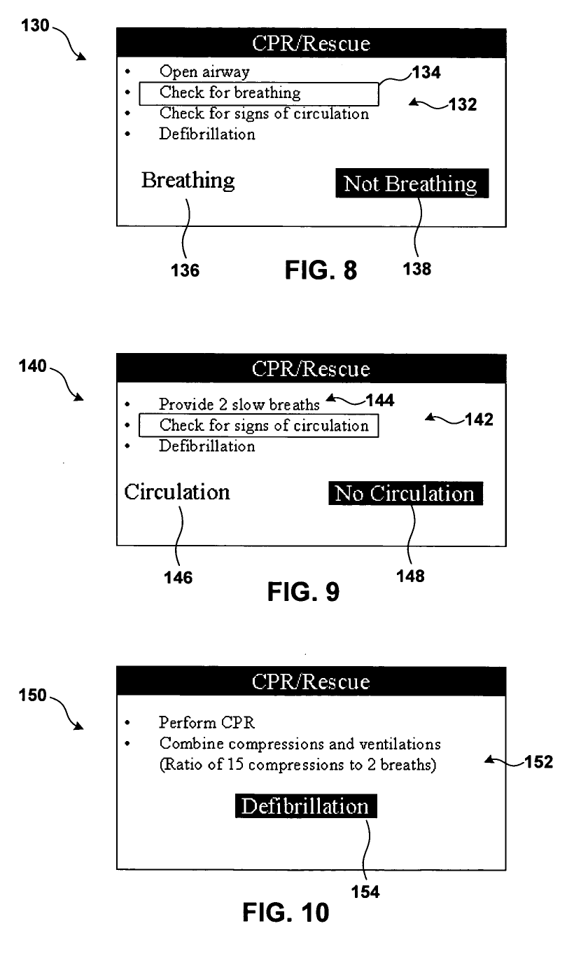

FIGS. 8 through 10 are exemplary screen displays 130, 140, 150 that illustrate presentation of information pursuant to a protocol, with branching. Branching pertains to presenting information pursuant to a protocol, with new or different information presented as a function of patient data.

In FIGS. 8 through 10, the presented information accompanies a CPR/rescue protocol. Screen display 130 shows a list of procedures 132 of a CPR/rescue protocol, i.e., open the airway of the patient, make sure the patient is breathing, check for signs of circulation and defibrillate the patient if necessary. An indicator such as a box 134 may highlight the action in progress.

The operator is prompted to report the status of an action. In FIG. 8, the operator, upon checking to be sure the patient is breathing, selects one of two prompts 136, 138 that best describes the condition of the patient. If the operator enters that the patient is breathing, then the device may prompt the operator to continue to the next step, i.e., checking the circulation of the patient. If the operator enters that the patient is not breathing, however, then the device may display new or different data.

FIG. 9 illustrates what may occur when the operator enters information indicating that the patient is not breathing. The list of procedures 142 may change to include a new procedure, i.e., an instruction to provide two slow breaths 144. The operator may be prompted to report the status of the patient following ventilation (not shown in FIG. 9), and may then proceed to the next action, checking for signs of circulation. The operator, after checking the circulation by, for example, checking the pulse or listening to the chest, selects one of two prompts 146, 148 that best describes the condition of the patient.

If the operator enters that the patient exhibits a lack of circulation, then the device may prompt the operator to perform CPR or to take other action. Screen display 150 in FIG. 10 illustrates that the list of procedures 152 has changed to include new procedures pertaining to administration of CPR. The operator may also be prompted to begin defibrillation procedures. When the operator has performed CPR and is ready to begin defibrillation procedures, the operator may select the defibrillation prompt 154. Selection of the defibrillation prompt may cause a defibrillation therapy protocol to be initiated and may cause a defibrillation-related screen display, such as screen display 90 shown in FIG. 5, to appear.

In response to prompts 146, 148 in screen display 140, the operator may select prompt 146, reporting that the patient has good circulation. In response to such a selection, displays of information pursuant to the CPR/rescue protocol may or may not be terminated, depending on the procedures of the protocol. If the CPR/rescue protocol is not terminated, the operator may be prompted to perform additional procedures such as administering a twelve-lead ECG, but the defibrillation procedure shown in list 142 will be aborted. If the CPR/rescue protocol is terminated, the operator may be presented with a screen display such as screen display 80 shown in FIG. 4.

FIGS. 8 through 10 demonstrate that the information displayed pursuant to one or more protocols may change, as new patient data are acquired. A protocol need not include a fixed list of procedures, but may be flexible and adaptive to the condition of the patient. A device that presents information pursuant to one or more protocols may, therefore, branch to display new or different procedures depending upon the condition of the patient.

Branching may also be used in diagnosing and treating medical conditions. When a patient presents the symptoms of a stroke, for example, device may present procedures that will help determine whether the stroke is ischemic or hemorrhagic. When an ischemic stroke is identified, the device may branch to a protocol directed to treatment of ischemic strokes, and when a hemorrhagic stroke is identified, the device may branch to a protocol directed to treatment of hemorrhagic strokes. Similarly, the device may branch when a potential diagnosis is ruled out, even if other diagnoses are possible.

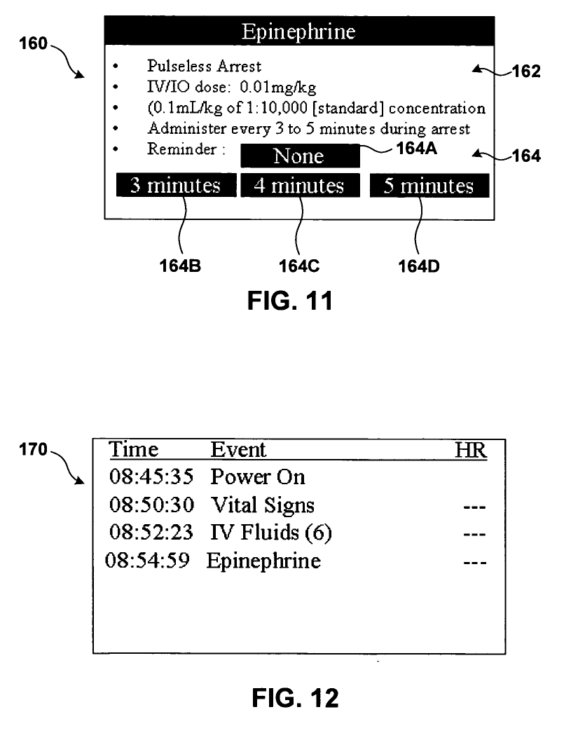

FIG. 11 is an exemplary screen display 160 that may accompany an epinephrine administration protocol. A patient exhibiting pulseless cardiac arrest may be treated with multiple dosages of epinephrine according to the protocol. The protocol may specify, for example, whether a dosage should be administered in a bolus or a drip, and may also specify a range of timing of the dosage administrations. Screen display 160 illustrates an exemplary epinephrine administration procedure 162, which specifies that epinephrine be administered every three to five minutes during arrest. The epinephrine protocol may therefore give the operator some discretion in administration.

Screen display 160 further shows that the operator is prompted to select a reminder time 164. Four reminder time options are presented: none 164A, three minute 164B, four minutes 164C and five minutes 164D. The operator selects the amount of time to pass between reminders. The selection by the operator may activate a timer that will prompt the operator to administer (or to consider administering) epinephrine after the selected time interval has passed. The operator may be reminded by a change in screen display, a pop-up notification, a tone, a buzzer, a voice prompt, or any other technique for attracting attention and reminding the operator to administer a new dosage.

Some medications may be administered in several dosages, but the number of dosages may be limited to avoid concerns about toxicity. When the operator has administered the final allowable dosage, the device may display a notice that the final allowable dosage had been administered, and that no further reminders will be given.

FIG. 12 shows an exemplary screen display 170 of an event log. An event log may include a report showing actions taken in the course of attending to patient 10. As shown in FIG. 12, an event log may include a description of the action and the time the action was taken. An event log may further include other data associated with the event, such as a monitored heart rate. An event log may be useful for emergency room personnel in a hospital who need to know the course of the pre-hospital treatment. An event log may also be helpful to an operator in preparing a run report.

In addition, an event log may be used to determine whether the operator followed an established protocol, or whether the operator had reason to depart from the established protocol. The effectiveness of a protocol itself may be analyzed by analysis of one or more event logs.

As noted above, protocols applicable to a given situation differ from jurisdiction to jurisdiction. Some regulating authorities may favor monitoring of every step of patient care, and may favor prompting the operator to report each action taken and may prefer generation of a detailed event log. Other regulating authorities may favor protocols in which significant events are prompted or recorded, and relatively minor events are not.

Regulating authorities may also establish protocols based upon the resources of their jurisdictions. For example, one jurisdiction may equip its emergency personnel with AEDs, while another jurisdiction may equip its emergency personnel with full-featured defibrillators. Because the defibrillation equipment in the jurisdictions differs, the regulating authorities for the respective jurisdictions may prescribe different defibrillation protocols.

Also, as already noted, a protocol applicable to an operator with minimal life-saving training may be quite different from a protocol applicable to an operator with extensive training. Further, protocols may be modified, improved or abandoned as techniques and equipment change, or as data pertaining to the effectiveness of a particular protocol accumulates. There may be innumerable reasons why protocols for the same complaint, condition or presentation may vary from place to place, from operator to operator, or from time to time.

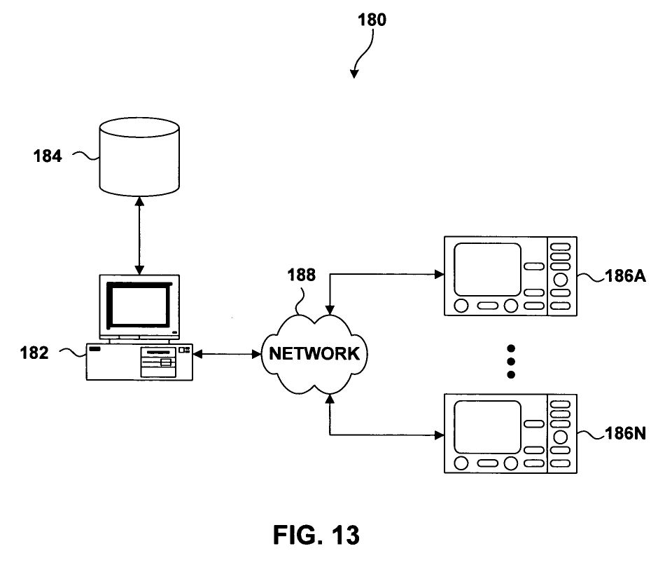

FIG. 13 is a schematic diagram of an exemplary system 180 for customizing a protocol. A regulating authority responsible for overseeing EMTs may operate a server 182 with local storage 184 that stores a plurality of protocols. Local storage may be any medium for storing computer-readable instructions or data, such as a magnetic or optical storage medium. In a typical application, it is usually more efficient to customize a protocol by updating an existing protocol than it is to generate an entirely new protocol. In other words, a typical customized protocol may represent a modified, expanded or otherwise updated version of an earlier protocol.

Server 182 retrieves a protocol from a storage site such as local storage 184. The retrieved protocol need not be stored locally, however. Server 182 updates the protocol according to directives of the regulating authority, and may save the customized protocol to local storage 184. Server 182 may further communicate the customized protocol to one or more client devices 186A-186N (hereinafter 186) via a network 188. Examples of client devices include defibrillator 12 and patient monitor 40. Network 188 may be any network, including a local network, the Internet, a telephone network or a wireless communication network.

Client devices 186 receive and store the customized protocol. When the device selects the customized protocol, the device presents information according to the customized protocol. In this way, a protocol may be customized by a centralized regulating authority and distributed to EMTs throughout a jurisdiction.

Server 182 may also collect data from devices 186 via network 188. The regulating authority may use data collected in this fashion to monitor the adherence to protocols and the effectiveness of protocols.

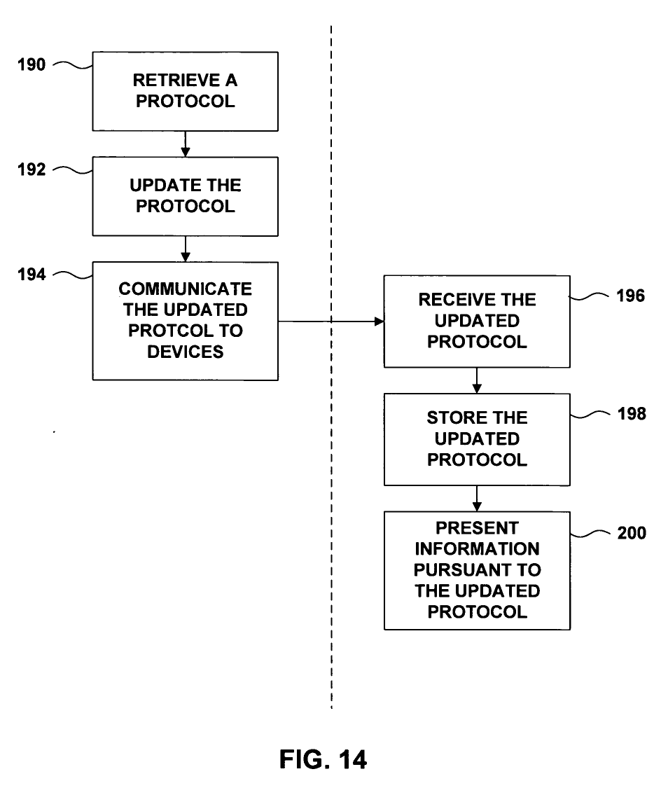

FIG. 14 is a flow diagram illustrating customization. Server 182 retrieves a protocol from a storage site (190) for customization. Server 182 may retrieve the protocol from local storage 184 or from another storage site. For example, server 182 may retrieve the protocol from a device or a remote database via network 188.

Server 182 updates the protocol according to directives of the regulating authority (192). Updating may include modifying, expanding or otherwise revising the protocol. The protocol may be stored on a computer-readable medium such as local storage 184, and may be tested and debugged with server 182. Server 182 communicates the customized protocol to client devices 186 (194).

Client devices 186 receive the customized protocol (196) and store the customized protocol (198). The customized protocol may be stored on computer-readable medium. Storing the customized protocol may include purging an older protocol and replacing the older protocol with the customized protocol. Storing the customized protocol may also include retaining the older protocol but incorporating updates to the older protocol. Storing the customized protocol may further include adding the customized protocol to older protocols stored on the device, without changing any older protocols. When used by an operator attending to a patient, device 186 may select the customized protocol and present information according to the customized protocol (200).

Protocols may be embodied in any of many computer-readable formats, such as linked list data structure or other data structure. In one embodiment, a protocol may be embodied in an object-oriented computer language and may include a set of objects related by links. The objects may be in embodied as instructions in an object-oriented computer language such as Java, C++ or ActiveX.

In general, objects include data and defined procedures for manipulating the data. Objects may include text, pictures, sounds and instructions for a processor. Screen displays 80, 90, 100, 110, 130, 140, 150 and 160 may be examples of representations of one or more objects. Representations of objects may include menus, information entry fields, pop-up notifications, prompts, icons, and so forth. A protocol may move from object to object by any of several links, such as by selection of prompts by an operator, by entry of data from an operator, or in response to data sensed from patient 10.

Customization of a protocol may be realized by customization of objects and the links among objects. Customization may be further realized by creation of new objects and removal of unneeded objects as deemed appropriate by the regulating authority. In addition, a new protocol may be developed from a library of standard objects or from objects used in other protocols.

Retrieving a protocol (190), therefore, may include retrieving one or more objects, or retrieving a set of objects related by links. Updating the protocol (192) may include amending the objects, selecting new objects, removing objects or changing the links relating the objects. Communication of the updated protocol (194) may include communicating the entire customized protocol or selected portions of the customized protocol, such as updated objects and links.

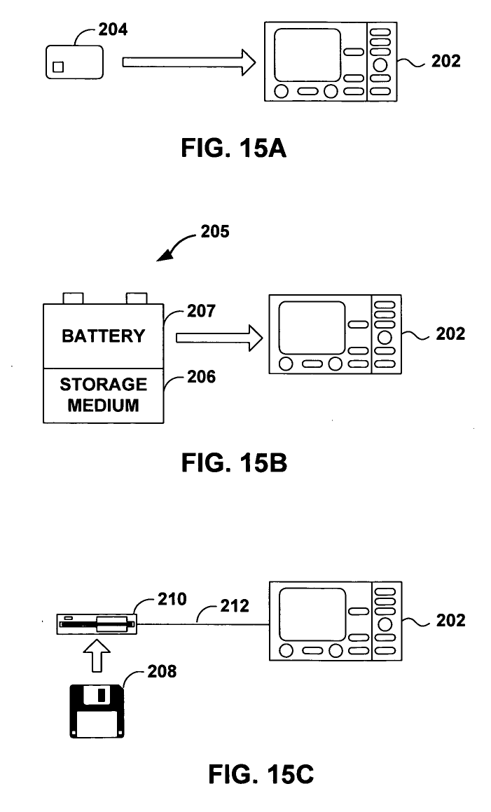

FIGS. 15A-15C illustrate additional techniques for updating health care protocols. The techniques depicted in FIGS. 15A-15C comprise receiving a recorded storage medium that includes a first health care protocol. An input device retrieves the first health care protocol from the medium. A medical device 202, having a second health care protocol in memory, can update the second protocol based on the first health care protocol.

In FIG. 15A, medical device 202 receives a recorded storage medium 204, which includes a health care protocol. Medical device 202 may be a defibrillator, a patient monitor, a trainer, or other medical device. In FIG. 15A recorded storage medium 204 is embodied as a data card or “smart card” and is received by an input device such as a built-in reader in medical device 202, but the invention is not limited to use of a data card. Recorded storage medium 204 may alternatively include a compact disk, a data tape, a floppy disk, a USB storage media, a hard disk, or the like.

Typically, the first health care protocol stored on recorded storage medium 204 is a newer version of the second health care protocol stored in the memory of medical device 202, and supplants, supercedes, or modifies the second protocol in one or more respects. Accordingly, medical device 202 receives the first health care protocol and updates the second health care protocol based on the first health care protocol.

FIG. 15B illustrates a storage media device 205 that includes a battery 207 coupled to a recorded storage medium 206. Battery 207 serves as a power supply for medical device 202, and may be configured to mate with medical device 202. As part of repair or routine maintenance, for example, medical device 202 may receive a new power supply. When receiving a new power supply, medical device 202 receives recorded storage medium 206 at the same time. Recorded storage medium 206 includes a first health care protocol. Medical device 202 receives the first health care protocol and updates a second health care protocol based on the first health care protocol. In other words, coupling storage media device 205 to medical device 202 not only replaces the power supply, but also comprises electronically coupling recorded storage medium 206 to a built-in reader or other input device that can retrieve the first health care protocol from recorded storage medium 206.

FIG. 15C illustrates a medical device 202 that receives a storage medium 208 via an input device that includes an adapter 210. Adapter 210 may be connected to medical device 202 via physical link 212. In FIG. 15C, adapter 210 comprises a disk drive configured to receive a floppy disk 208. The invention encompasses use of other storage media and adapters, such as a compact disk and an optical disk drive, a data tape and a tape drive, a data card and a reader, and the like.

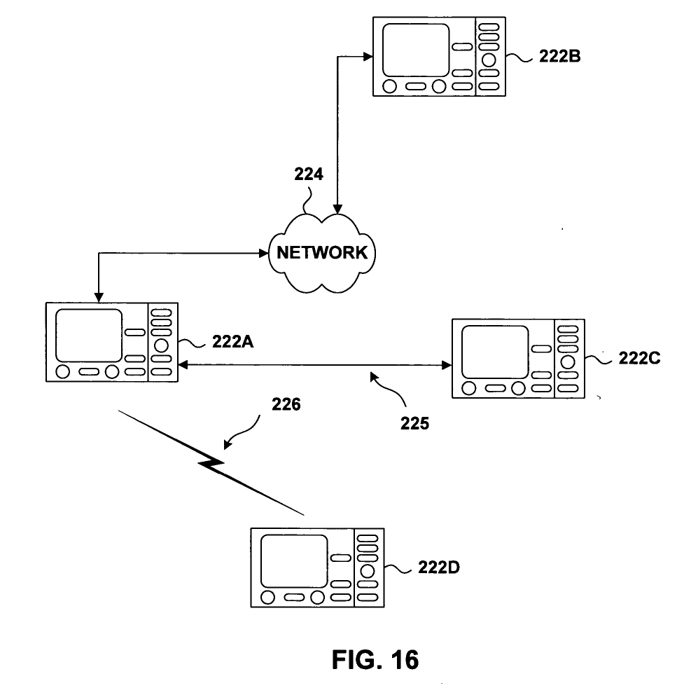

FIG. 16 depicts illustrative techniques by which a medical device can receive an updated health care protocol from another medical device. In FIG. 16, medical device 222A supplies or transmits the first health care protocol, and medical devices 222B, 222C and 222D receive the first health care protocol from medical device 222A.

Medical devices 222B, 222C and 222D are configured to receive the first health care protocol from medical device 222A in various ways. In one configuration, medical device 222A transmits the health care protocol to medical device 222B over a network 224. Network 224 may be any network, including a local network, the Internet, a telephone network, a cellular telephone network or a wireless communication network.

In another configuration, medical device 222A transmits the health care protocol to medical device 222C over a direct communication link 225. Communication link 225 may be a coaxial cable or any other type of physical communication link. In one embodiment, medical devices 222A and 222C are simultaneously coupled to a patient, and medical devices 222A and 222C communicate by using the body of the patient as a communication medium.

In a further configuration, medical device 222A transmits the health care protocol to medical device 222D over a wireless connection 226. Wireless connection 226 may make use of any wireless technology or wireless communication techniques. One communication protocol, commonly referred to as Bluetooth, uses short-range 2.4 GHz radio technology employed to transport data between devices. Other possible communication protocols include IEEE 802.11a, 802.11b, and 802.11g, which are industry standard protocols for wireless networking. Wireless connection 226 may also comprise radio frequency, infrared-based techniques, or the like.

The communications techniques depicted in FIG. 16 are not limited to like devices. Medical device 222A may be a defibrillator, for example, and medical devices 222B, 222C and 222D may include a chest thumper, a patient monitor and a trainer. Furthermore, a single medical device can supply the first health care protocol to one or more medical devices at a time.

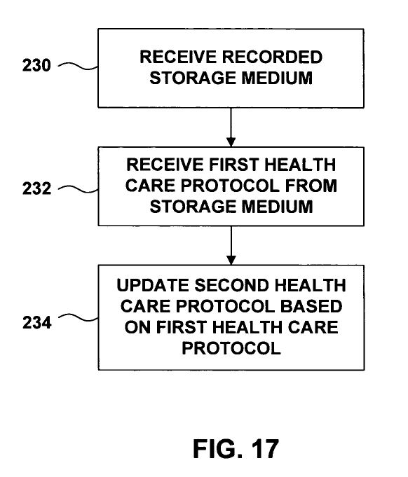

FIG. 17 is a flow diagram illustrating an exemplary technique for using a recorded storage medium to update a health care protocol. A medical device, such as a defibrillator, patient monitor or trainer, receives a recorded storage medium (230), such as a data card, a compact disk, a data tape, a floppy disk, a USB storage media, a hard disk, or the like. The recorded storage medium may be coupled to the medical device directly as shown in FIG. 15A or via an adapter, such as a disk drive, as shown in FIG. 15C. The data storage device may also be coupled to a battery as shown in FIG. 15B.

The medical device receives a first health care protocol from the storage medium (232) and updates a second health care protocol based on the first health care protocol (234). In a typical implementation of the technique shown in FIG. 17, the second health care protocol may be programmed onto the medical device or stored in the memory of the medical device before receipt of the storage medium, and the first protocol comprises an updated version of the second health care protocol. By updating the second health care protocol based on the first health care protocol (234), the medical device can implement an updated version of an earlier protocol. The medical device can, for example, present the operator with a task to be performed pursuant to the updated protocol and receive an acknowledgement from the operator that the task has been performed.

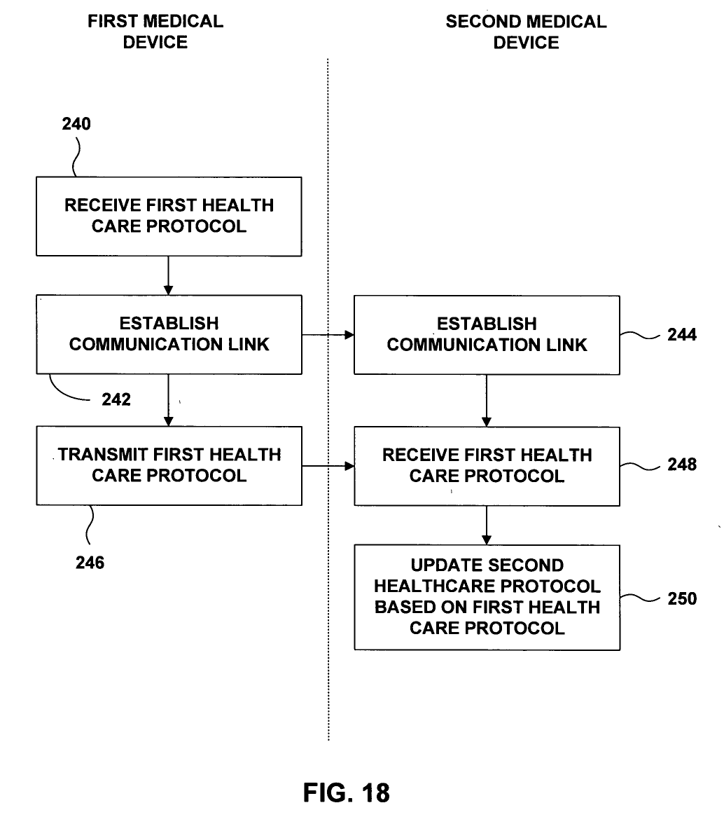

FIG. 18 is a flow diagram illustrating an exemplary technique for communicating an updated health care protocol between or among medical devices. A first medical device receives a first health care protocol (240), and may receive the protocol according to techniques such as those depicted in FIGS. 13-17. The first medical device establishes a communication link with a second medical device (242, 244), according to techniques such as those illustrated in FIG. 16. Upon establishment of the communication link, the first medical device transmits the first health care protocol (246), and the second medical device receives the first health care protocol from the first medical device via the established communication link (248). The second medical device may update a second health care protocol based on the first health care protocol (250).

The first medical device likewise may update a previously stored health care protocol based on the first health care protocol, and in this way, the first and second medical devices can implement an updated version of a protocol. When an updated version of the health care protocol is created, one medical device may receive the updated version and supply the updated version to other medical devices.

The invention may have one or more advantages. Emergency medical personnel may respond to a wide variety of emergencies, and it may be difficult to remember the protocols for all of the situations the personnel may encounter. The invention helps guide the emergency medical personnel through the established procedures of an applicable protocol. Moreover, the invention providing techniques for updating protocols for medical devices, including trainers, medical devices can help emergency medical personnel apply the current protocols.

Updating a health care protocol by various means allows a medical device to be flexible and conform to protocols of different jurisdictions. As the protocols of different jurisdictions change, the protocols stored in a plurality of medical devices can be readily updated. Moreover, the updates to a health care protocol may be supplied to a medical device via many paths.

Although the invention can be advantageous to emergency medical personnel , the invention is not limited to devices used by emergency medical personnel. The invention can be useful to any health care professional or any emergency responder.

The preceding specific embodiments are illustrative of the practice of the invention. Various modifications may be made without departing from the scope of the claims. For example, the invention need not be embodied in a medical device such as defibrillator or medical monitor. The invention may be embodied in a stand-alone device that provides no diagnosis, monitoring or therapy. The device may be small and easily portable, but the invention is not limited to application with small, portable devices. Nor is the invention limited to medical devices. The invention may also be embodied in a device that performs functions other than medical functions, such as a personal digital assistant or a cellular telephone. The invention may also be embodied in a trainer that simulates a medical device, as well as a working medical device that supports a training mode.

Furthermore, none of the updating techniques of the invention is exclusive of others. A medical device may, on one occasion, receive an update from another medical device. On another occasion, the medical device may receive an update via a recorded storage medium.

The invention is advantageous for emergency medical personnel working in the field, but the invention is not limited to that environment. Embodiments of the invention may be used in a hospital environment as well. The invention may assist emergency medical personnel working in a hospital emergency room, for example. These and other embodiments are within the scope of the following claims.

1. A method comprising:
establishing a communication link with a medical device;
receiving a first health care protocol from the medical device; and
updating a second health care protocol based on the first health care protocol.

2. The method of claim 1, wherein the communication link includes one of a wireless communication link.

3. The method of claim 1, wherein the medical device includes at least one of a defibrillator, a patient monitor, and a trainer.

4. The method of claim 1, wherein the first health care protocol includes a training health care protocol.

5. The method of claim 1, wherein the second protocol includes a set of procedures for treating the patient.

6. The method of claim 1, wherein the second protocol includes at least one of timers, checklists, prompts, display modes, references and utilities.

7. The method of claim 1, further comprising:
presenting the operator with a task to be performed pursuant to the second protocol; and
receiving an acknowledgement from the operator that the task has been performed.

8. The method of claim 1, wherein updating the second health care protocol comprises supplanting at least a part of the second health care protocol with a part of the first health care protocol.

9. A method comprising:
receiving a recorded storage medium;
receiving a first health care protocol from the recorded storage medium; and
updating a second health care protocol based on the first health care protocol.

10. The method of claim 9, wherein the recorded storage medium includes a battery.

11. The method of claim 9, wherein the recorded storage medium includes a data card.

12. The method of claim 9, wherein the second health care protocol includes a training health care protocol.

13. The method of claim 9, wherein the second protocol includes a set of procedures for treating the patient.

14. The method of claim 9, wherein the second protocol includes at least one of timers, checklists, prompts, display modes, references and utilities.

15. The method of claim 9, further comprising:
presenting the operator with a task to be performed pursuant to the second protocol; and
receiving an acknowledgement from the operator that the task has been performed.

16. The method of claim 9, wherein updating the second health care protocol comprises supplanting at least a part of the second health care protocol with a part of the first health care protocol.

17. A computer-readable medium comprising instructions for causing a programmable processor to:
establish a communication link with a medical device;
receive a first health care protocol from the medical device; and
update a second health care protocol based on the first health care protocol.

18. The medium of claim 17, wherein the communication link includes one of a wireless communication link.

19. The medium of claim 17, wherein the medical device includes at least one of a defibrillator, a patient monitor, and a trainer.

20. The medium of claim 17, wherein the first health care protocol includes a training health care protocol.

21. The medium of claim 17, wherein the second protocol includes a set of procedures for treating the patient.

22. The medium of claim 17, wherein the second protocol includes at least one of timers, checklists, prompts, display modes, references and utilities.

23. The medium of claim 17, further comprising instructions for causing the programmable processor to:
present the operator with a task to be performed pursuant to the second protocol; and
receive an acknowledgement from the operator that the task has been performed.

24. The medium of claim 17, wherein updating the second health care protocol comprises supplanting at least a part of the second health care protocol with a part of the first health care protocol.

25. A computer-readable medium comprising instructions for causing a programmable processor to:
receive a recorded storage medium;
receive a first health care protocol from the recorded storage medium; and
update a second health care protocol based on the first health care protocol.

26. The medium of claim 25, wherein the recorded storage medium includes a battery.

27. The medium of claim 25, wherein the recorded storage medium includes a data card.

28. The medium of claim 25, wherein the second health care protocol includes a training health care protocol.

29. The medium of claim 25, wherein the second protocol includes a set of procedures for treating the patient.

30. The medium of claim 25, wherein the second protocol includes at least one of timers, checklists, prompts, display modes, references and utilities.

31. The medium of claim 25, further comprising instructions for causing the programmable processor to:
present the operator with a task to be performed pursuant to the second protocol; and
receive an acknowledgement from the operator that the task has been performed.

32. The medium of claim 25, wherein updating the second health care protocol comprises supplanting at least a part of the second health care protocol with a part of the first health care protocol.

33. A system comprising:
a server to customize a health care training protocol; and
a client device to receive the customized health care training protocol over a network.

34. The system of claim 33, wherein the health care training protocol is used by a trainer to simulate the behavior of a defibrillator.

35. The system of claim 33, further comprising a storage site to store a plurality of protocols.

36. The system of claim 35, wherein the protocol comprises at least one of an object in an object-oriented language and a set of objects in an object-oriented language related by links.

37. The system of claim 33, wherein the network comprises at least one of a local network, an Internet network, a telephone network and a wireless communication network.

38. The system of claim 33, wherein the server collects data from the client device over the network.

39. An apparatus comprising:
a battery; and
a recorded storage medium that stores at least a part of a health care protocol.

40. The apparatus of claim 39, wherein the apparatus is configured to mate to at least one of a defibrillator, a patient monitor, and a trainer.

41. A medical device comprising:
an input device to receive recorded storage medium and to retrieve a health care protocol stored on the medium; and
a memory to store the health care protocol.

42. The device of claim 41, wherin the health care protocol is a first health care protocol and wherein the memory is further configured to store a second health care protocol, the device further comprising a processor to update the second health care protocol based on the first health care protocol.

43. The device of claim 41, wherein the input device comprises an adapter.

44. The device of claim 41, wherein the input device comprises a data card reader.

45. The device of claim 41, wherein the recorded storage medium includes a battery.
